(19) United States
(12) Patent Application Publication
(10) Pub. No.: US 2024/0008739 A1
(43) Pub. Date: Jan. 11, 2024

(54) OCT DEVICE

(71) Applicant: NIDEK CO, LTD., Gamagori-shi (JP)

(72) Inventors: Hirofumi YOGO, Gamagori-shi (JP); Seiji Masegi, Gamagori-shi (JP)

(73) Assignee: NIDEK CO., LTD., Gamagori-shi (JP)

(21) Appl. No.: 18/460,111

(22) Filed: Sep. 1, 2023

Related U.S. Application Data
(63) Continuation of application No. PCT/JP22/08184, filed on Feb. 28, 2022.

(30) Foreign Application Priority Data
Mar. 3, 2021 (JP) 2021-033935
Sep. 30, 2021 (JP) 2021-162174

(51) Int. Cl. A61B 3/10; A61B 3/107; A61B 3/12; A61B 3/117

(52) U.S. Cl. CPC A61B 3/102; A61B 3/107; A61B 3/1216; A61B 3/117

(57) ABSTRACT

An OCT device includes an OCT optical system, a controller which acquires OCT data based on a signal from the OCT optical system and controls the OCT optical system to execute an OCT data acquisition operation, a light guide optical system, and an adjuster adjusting a three-dimensional position of the light guide optical system with respect to a subject eye. The light guide optical system includes an optical scanner scanning the subject eye with the measurement light, and an objective optical system which is disposed between the optical scanner and the subject eye and forms a pivot point around which the measurement light passing through the optical scanner is pivoted. The controller guides the three-dimensional position such that the pivot point is arranged at a target position set between the subject eye and the objective optical system, and further executes the OCT data acquisition operation at the target position.

### CROSS-REFERENCE TO RELATED APPLICATIONS

This is a continuation of International Application No. PCT/JP2022/008184 filed on Feb. 28, 2022, and claims priority from Japanese Patent Applications No. 2021-033935 filed on Mar. 3, 2021 and No. 2021-162174 filed on Sep. 30, 2021, the entire content of which is incorporated herein by reference.

### TECHNICAL FIELD

The present disclosure relates to an OCT device.

### BACKGROUND ART

In the field of ophthalmology, an optical coherence tomography (OCT), which is a device for capturing a tomographic image of a tissue of a subject eye, is known.

In the technical field of OCT, various attempts are made to improve penetrability of OCT data (that is, to expand an imaging range in a depth direction).

In recent years, it has been reported that the imaging range can be remarkably improved by improving a light source. For example, adopting a light source called VCSEL that emits light with a long coherence length as an OCT light source is effective in improving penetrability. In deep-penetration OCT, it is also proposed to image a depth region from a cornea to a fundus in a single A-scan (see JP2015-157182A).

Also, in general, a fovea centralis on a retina is not on an axis of the eye (or an optical axis of the eye), but slightly eccentric to the temporal side. Therefore, a so-called physiological squint angle exists even in a normal ocular optical system. Various angles such as α angle, γ angle, κ angle, and λ angle are known as an index indicating the angle. For example, in recent years, these angles have been considered in situations such as the prescriptions of premium IOL (see JP2015-157182A). For example, in JP2015-157182A, the κ angle is obtained based on information on a front image of an anterior segment.

An OCT device obtains OCT data (B-scan data or volume data) according to a trajectory of measurement light by scanning a tissue of a subject eye with the measurement light by using an optical scanner. Meanwhile, no investigation is made on a scanning method that facilitates extensive scanning of both an anterior segment and a fundus of the subject eye in JP2015-157182A.

In addition, in the method adopted in JP2015-157182A and the like as a method for obtaining a physiological squint angle of a subject eye, an actual position of the fovea centralis is unknown. It is possible that wide-range OCT data provides a more appropriate physiological squint angle of the subject eye.

### SUMMARY OF INVENTION

A technical object of the present disclosure is to provide an OCT device that facilitates extensive scanning of both an anterior segment and a fundus of a subject eye.
(1) An OCT device having:
an OCT optical system including:
   a light splitter that splits light from an OCT light source into measurement light and reference light; and
   a detector that detects a spectral interference signal between the measurement light guided to a subject eye and the reference light;
an arithmetic controller that acquires OCT data based on a signal from the detector, and controls at least the OCT optical system to execute an OCT data acquisition operation;
a light guide optical system including at least:
   an optical scanner that scans a tissue of the subject eye with the measurement light; and
   an objective optical system that is disposed between the optical scanner and the subject eye, and forms a pivot point around which the measurement light passing through the optical scanner is pivoted; and
an alignment adjuster that adjusts a three-dimensional position of the light guide optical system with respect to the subject eye,
in which the arithmetic controller guides the three-dimensional position such that the pivot point is arranged at a target position set between the subject eye and the objective optical system, and further executes the OCT data acquisition operation at the target position.

The present disclosure facilitates extensive scanning of both an anterior eye and a fundus.

### BRIEF DESCRIPTION OF DRAWINGS

Exemplary embodiment(s) of the present invention will be described in detail based on the following figures, wherein:

FIG. 1 is a diagram illustrating a schematic configuration of an OCT system according to an example;

FIG. 2 is a diagram illustrating an OCT optical system according to the example;

FIG. 3 is a flowchart illustrating an imaging operation;

FIG. 4A is a diagram illustrating a positional relationship between a device and a subject eye E in a fundus mode;

FIG. 4B illustrates fundus OCT data acquired in the fundus mode;

FIG. 5A is a diagram illustrating a positional relationship between the device and the subject eye E in an anterior segment mode;

FIG. 5B illustrates fundus OCT data acquired in the anterior segment mode;

FIG. 6A is a diagram illustrating a positional relationship between the device and the subject eye E in a whole-eye mode;

FIG. 6B is a diagram illustrating fundus OCT data acquired in the whole-eye mode;

FIG. 7 is a diagram illustrating synthetic OCT data;

FIG. 8 is a diagram for explaining analysis processing; and

FIG. 9 is a diagram illustrating a measurement method according to a modification example.

### DESCRIPTION OF EMBODIMENTS

(Outline)

An embodiment of the present disclosure will be described. Items classified by < > below may be used independently or in relation to each other. An OCT device according to each embodiment is appropriate for acquiring OCT data over a wide area.

### First Embodiment

An OCT device according to a first embodiment includes at least an OCT optical system, a light guide optical system, an arithmetic controller, and an alignment adjuster.

<OCT Optical System>

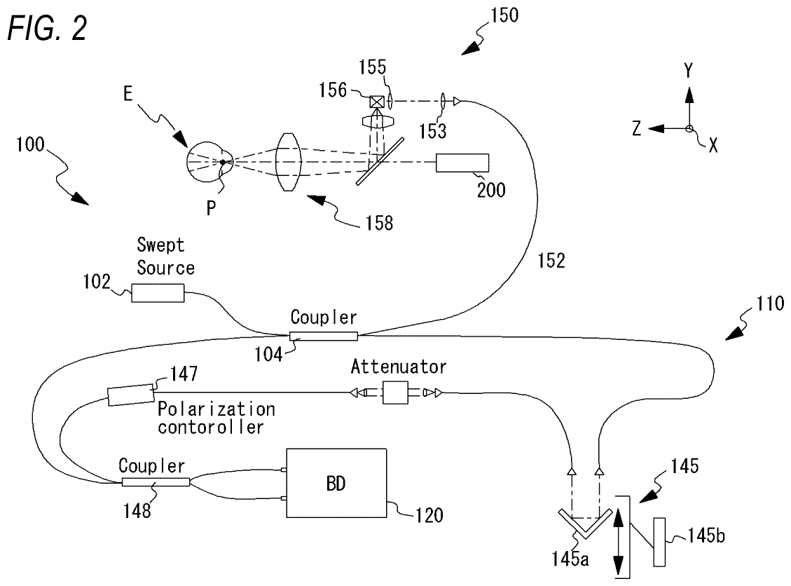

The OCT optical system (refer to FIG. 2) is used to capture an image of OCT data of a subject eye. The OCT optical system has at least a light splitter and a detector. The light splitter is used to split light from an OCT light source into measurement light and reference light. The detector detects a spectral interference signal of the measurement light and the reference light guided to the subject eye. The OCT data is acquired by processing the signal from the detector by the arithmetic controller, which will be described later.

The OCT optical system may be appropriate for acquiring the OCT data with high penetrability (in other words, over a wide area). For example, the OCT optical system according to the first embodiment may be a wavelength-swept OCT (SS-OCT) optical system. In this case, the OCT optical system includes a wavelength-swept light source (wavelength scanning light source) as an OCT light source that is a light source for measurement light and reference light. The wavelength-swept light source changes an emission wavelength at high speed in time. For example, since a VCSEL-type wavelength-swept light source has a long coherence length, it can be used as an OCT light source to capture OCT data over a wide range in a depth direction. For example, an imaging range of approximately 10 mm or more can be achieved. Thus, a plurality of tissues at different depth positions in the subject eye can be imaged at once. As a specific example, both a fundus and a translucent body can be imaged at once. Further, it is preferable that the wavelength-swept light source performs wavelength sweeping in a so-called 1 μm band (wavelength sweeping is performed centering on approximately 1050 nm). It is known that the so-called 1 μm band exhibits higher penetrability into the tissues of the subject eye than other wavelength bands.

A sweep frequency in the wavelength-swept light source may be changeable between at least a first frequency and a second frequency. The second frequency has a smaller value than a value of the first frequency. For example, the sweep frequency is changed by changing a speed of an optical element built in the light source and driven to sweep a wavelength or a duty ratio in a sweep cycle.

<Converter>

In a case where the optical system of the device is an SS-OCT optical system, the OCT device further includes a converter. In the SS-OCT optical system, the detector detects a spectral interference signal as a beat signal, as a wavelength is swept. The converter samples the spectral interference signal output from the detector. Further, the converter converts the spectral interference signal output from the detector from an analog signal to a digital signal. The converter may be a digitizer capable of adjusting a sampling frequency.

<Light Guide Optical System>

The light guide optical system forms at least a part of a measurement optical path for guiding measurement light to the subject eye. More specifically, the light guide optical system of the present embodiment includes at least an optical scanner and an objective optical system. The optical scanner scans measurement light on a tissue of the subject eye. For example, the light guide optical system may be provided with two optical scanners having different scanning directions. Further, the objective optical system is arranged between the optical scanner and the subject eye. Thus, the objective optical system forms a pivot point for the measurement light. The measurement light passing through the optical scanner is pivoted centering on the pivot point.

Here, the measurement light passing through the pivot point is scanned along a plurality of predetermined scan lines on the tissue of the subject eye. OCT data for each scan line is captured along with the scanning. The scan line may be set at any position based on an instruction from an examiner. Further, a scan line corresponding to a scan pattern may be set by selecting any of a plurality of predetermined scan patterns. Various scan patterns, such as line, cross, multi, map, radial, and circle, are known.

<Alignment Adjuster>

The alignment adjuster adjusts a three-dimensional position of the light guide optical system with respect to the subject eye. At this time, in the present embodiment, at least a position of the light guide optical system in a forward-rearward direction with respect to the subject eye is adjusted. The light guide optical system may be electrically moved by an actuator provided in the alignment adjuster. The alignment adjuster is not limited to this, and may be a mechanical mechanism. Further, the alignment adjuster may include a face support unit capable of changing a position of a face of a subject person. In other words, the three-dimensional position of the subject eye may be adjusted by moving the position of the face of the subject person.

The OCT device according to the present embodiment may additionally have an alignment detection optical system. The alignment detection optical system is used to guide the light guide optical system to an appropriate operation distance with respect to the subject eye. The alignment detection optical system detects an alignment state of the light guide optical system with respect to the subject eye at least in a Z-direction. In the present embodiment, the alignment detection optical system may include at least an observation optical system (preferably an anterior segment observation optical system). In this case, the alignment detection optical system may further include a light projecting optical system for projecting an operation distance detection index onto an anterior segment of the subject eye. The operation distance may be adjusted based on a position of the index or an imaging state observed by the observation optical system. Further, the OCT optical system may be used as the alignment detection optical system. In this case, the alignment state may be adjusted based on OCT data such that an image of the anterior segment is captured at a predetermined position on the OCT data.

<Arithmetic Controller>

The arithmetic controller acquires OCT data based on a signal from the detector. More specifically, a spectral interference signal converted into a digital signal by the converter is arithmetically processed by an image processor. Thus, the OCT data of the subject eye is acquired. Further, the arithmetic controller controls at least the OCT optical system to execute an OCT data acquisition operation.

<OCT Data>

OCT data may be signal data, or may be visualized image data. For example, the OCT data may be at least any one of tomographic image data indicating reflection intensity characteristics of the subject eye, OCT angio-data of the subject eye (for example, OCT motion contrast data), Doppler OCT data indicating Doppler characteristics of the subject eye, polarization characteristic data indicating polarization characteristics of the subject eye, or the like.

The OCT data may be at least any one of B-scan data (for example, B-scan tomographic image data, two-dimensional OCT angio-data, and the like), front (En face) data (for example, OCT front data, front motion contrast data, and the like), three-dimensional data (for example, three-dimensional tomographic image data, three-dimensional OCT angio-data, and the like), or the like.

<Application of Full-Range Technique>

A full-range technique may be applied to OCT data. Various methods for removing virtual images in the OCT data are called full-range techniques. In the present embodiment, any full-range technique may be applied, which may allow acquisition of a wider range of OCT data from which virtual images have been selectively removed.

As an example of the full-range technique, a technique of removing a virtual image (also referred to as a mirror image) by additional hardware (see, for example, Non-patent Literature 2 shown below), a technique of correcting by software without using additional hardware (see, for example, JP2015-506772A), and the like can be mentioned.
Non-patent Literature 1: Cecilio Velasco-Barona et al, “Personalized Optical Designs and Manipulating Optics: Applications on the Anterior Segment of the Eye” Journal of Ophthalmology, 27 Nov. 2019 (Published)
Non-patent Literature 2: Wojtkowski, M. et al. (2002) Full range complex spectral optical coherence tomography technique in eye imaging, Optics Letters, 27 (16), p. 1415.

Further, in an application by the present applicant (Japanese Patent Application No. 2019-014771) disclosed in JP2020-022723A, another full-range technique has been proposed as follows. Based on a plurality of pieces of OCT data having different optical path lengths when detecting a spectral interference signal, at least complementing processing is performed on an overlapping region between a real image and a virtual image in the OCT data, and the OCT data subjected to the complementing processing is generated, and this may be applied to the present embodiment.

<Position Guidance of Light Guide Optical System>

For example, the arithmetic controller guides a three-dimensional position of the light guide optical system with respect to the subject eye such that a pivot point is arranged at a target position. In a case where the pivot point reaches the target position as a result of the guidance, the OCT data acquisition operation is executed.

The position guidance of the light guide optical system for arranging the pivot point at the target position may be so-called auto-alignment. That is, the light guide optical system may be moved in a direction in which a deviation between a position of the pivot point and the target position is reduced, by driving and controlling the alignment adjuster by the arithmetic controller. Alternatively, or in addition to the auto-alignment, guide information for assisting alignment may be output to the examiner such that the pivot point is arranged at the target position. The guide information may be graphical information displayed on a monitor (for example, character information, graphic information, and the like, details of which will be described later), or audio information output from a speaker. For example, the guide information may be operation guidance for the examiner.

In the present embodiment, the target position of the pivot point is set between the subject eye and the objective optical system. The target position may be set at a position a predetermined distance away from a corneal vertex in a forward-rearward direction.

Since the target position is between the subject eye and the objective optical system, measurement light that is not parallel to an optical axis is incident on a cornea of the subject eye while moving away from an optical axis of the optical system. Such measurement light is guided to each tissue of the anterior segment of the eye and the fundus without crossing the optical axis again within an eyeball. Thus, it is possible to irradiate a wider range of the subject eye with the measurement light.

Here, a difference in measurement range between two comparative examples (Comparative Examples 1 and 2) in which the measurement light is guided to the subject eye without the pivot point being formed between the subject eye and the objective optical system and the present embodiment will be described.

In Comparative Example 1, a pivot point is arranged inside an eyeball of a subject eye. In Comparative Example 1, a portion of the subject eye at a depth position near the pivot point is less likely to be irradiated with measurement light, and is less likely to be imaged. On the other hand, in the present embodiment, since the pivot point is not arranged in the eyeball of the subject eye by being set between the subject eye and the objective optical system, a tissue at each depth position is likely to be imaged, as compared with Comparative Example 1.

In Comparative Example 2, a subject eye is irradiated with measurement light substantially telecentrically from an objective optical system. Since a telecentric luminous flux is refracted by a translucent body of the subject eye, the measurement light reaching a fundus is concentrated in an approximate center of the fundus (near a fovea centralis). Therefore, in Comparative Example 2, it is difficult to ensure an imaging range on the fundus. On the other hand, in the present embodiment, the measurement light is incident on the cornea of the subject eye from the pivot point between the subject eye and the objective optical system while moving away from the optical axis of the optical system. Therefore, even in a case where the measurement light is refracted by the translucent body of the subject eye, the measurement light is likely to be emitted to (that is, can be imaged) a position distant from the approximate center of the fundus (near the fovea centralis).

Here, in the OCT device according to the present embodiment, the scanning amount of the measurement light may be set such that the measurement light is intentionally eclipsed by an iris at the target position. In this case, as a result of executing the OCT data acquisition operation at the target position, OCT data including at least the cornea, the fundus, and the iris can be acquired once (in other words, in one shot).

Since the OCT data acquired in one shot includes positional information on the cornea, the iris, and the fundus, it is possible to appropriately specify a positional relationship between the fundus and the anterior segment of the eye. In addition, since the positional relationship between the fundus and the anterior segment can be appropriately specified, the OCT data can be appropriately synthesized (collaged) with the local OCT data of the anterior segment or the fundus.

<Adjustment of Measurement Range in Depth Direction>

The OCT device according to the present embodiment may include a second adjuster. The measurement range in the depth direction in the OCT data may be adjustable (changeable) by controlling the second adjuster by the arithmetic controller. For example, the measurement range in the depth direction may be switchable between at least a first measurement range and a second measurement range that is narrower than the first measurement range based on the control of the second adjuster.

For example, in a case of SS-OCT, one or both of the wavelength-swept light source and the converter may be used as the second adjuster. In this case, a width of the measurement range in the depth direction in OCT data is changed by changing one or both of a sweep frequency and a sampling period of an interference signal. In the present embodiment, at least in a case where the OCT data acquisition operation is executed at the target position set between the subject eye and the objective optical system, the measurement range may be adjusted to include the cornea to the fundus. More specifically, the width of the measurement range in the depth direction may be adjusted such that the measurement range is larger than an axial length of the subject eye.

Further, the second adjuster may include an optical path length difference adjuster. The optical path length difference adjuster changes at least one of an optical path length of the measurement light and an optical path length of the reference light. Thus, the optical path length difference adjuster adjusts an optical path length difference between the measurement light and the reference light. The measurement range is displaced in the depth direction according to the optical path length difference. The optical path length difference adjuster may be a device that changes an optical path length of at least any one of an optical path on an upstream side (light source side) of the objective optical system in the light guide optical system or an optical path of a reference optical system.

Further, the OCT device according to the present embodiment may have a focus adjuster that adjusts a focus position of the measurement light.

The arithmetic controller may acquire a plurality of pieces of OCT data having mutually different focus positions of the measurement light at a target position where the pivot point is arranged between the subject eye and the objective optical system, and synthesize the plurality of pieces of OCT data to acquire synthetic OCT data. Thus, even in a case where a depth of field of the OCT optical system is not sufficiently large relative to the axial length of the eye, it becomes easier to acquire OCT data with overall high luminance.

### Second Embodiment

Next, a second embodiment will be described. An ophthalmic image processing program according to the second embodiment is executed by a processor of an ophthalmic computer to cause the ophthalmic computer to execute an ophthalmic image processing method according to following each step. The ophthalmic computer may be integrated with an OCT device, or may be a separate body. In a case where the ophthalmic computer is the separate body, the ophthalmic computer and an ophthalmic imaging apparatus are connected in a wire or wireless manner, and can communicate with each other.

For convenience of description, unless otherwise noted, the ophthalmic computer is integrated with the OCT device in the description of the embodiments and examples. In this case, the ophthalmic image processing program is executed by the arithmetic controller described above.

<Acquisition Step>

In the second embodiment, the arithmetic controller may acquire wide-area OCT data of a subject eye. The wide-area OCT data includes at least OCT data of an anterior segment and OCT data of a fundus. The OCT data of the fundus is acquired in a state in which a pivot point of measurement light is arranged on a side of the subject eye relative to the objective optical system. As a result, the wide-area OCT data in the present embodiment has a sufficient amount of information for specifying optical features of the subject eye in a transverse direction of the fundus.

The wide-area OCT data may be captured in one shot by the imaging method described in the first embodiment. Alternatively, one-shot imaging may be performed in a state in which the pivot point is arranged within an eyeball of the subject eye.

<Analysis Step>

By analyzing wide-area OCT data (for example, B-scan data acquired at the target position in the first embodiment), the arithmetic controller may acquire information representing a tilt of the anterior segment with respect to the fundus (hereinafter, referred to as tilt information).

For example, by analyzing B-scan data including the cornea, the iris, and the fundus as the wide-area OCT data, information representing the tilt of the anterior segment with respect to the fundus may be acquired. In this case, for example, the tilt information may be acquired based on a positional relationship between a pupil center and a fovea centralis on the fundus. The pupil center may be a center of an end portion of the iris, particularly preferably a center of an outer end portion of the iris (which may be a corner angle position).

As an example, a straight line connecting the pupil center and the fovea centralis may be acquired as the tilt information. The straight line itself, or the amount of deviation between the straight line and a reference axis of the device or the subject eye may be acquired as the tilt information.

The amount of deviation may be obtained, for example, as an angle between the straight line connecting the pupil center and the fovea centralis and an optical axis of the OCT optical system. For example, it is possible that more appropriate IOL prescriptions or the like can be proposed based on the tilt information.

Further, as the tilt information, a physiological squint angle of the subject eye may be obtained. The physiological squint angle includes, for example, α angle, γ angle, κ angle, λ angle, and the like. Any of these physiological squint angles may be acquired by analyzing wide-area OCT data.

In a case where the wide-area OCT data includes the cornea, the crystalline lens, and the fovea centralis on the fundus, the physiological squint angle can be obtained, based on the cornea, the crystalline lens, and the fovea centralis on the fundus in the wide-area OCT data.

Here, a case will be described where, for example, the κ angle, which is one of the physiological squint angles, is measured by using wide-area OCT data captured in a state in which a pivot point of measurement light is adjusted to the anterior segment of the subject eye. The κ angle is defined as an angle between a visual axis of the subject eye and a pupil centerline, and the visual axis is approximated by an A-scan passing through the fovea centralis on the retina. Further, an optical axis of the eye is specified from information on the cornea and the crystalline lens in the OCT data. For example, from shape information on the cornea and the crystalline lens in the OCT data, an A-scan passing through curvature centers of the cornea and the crystalline lens (more specifically, curvature centers of front and back surfaces of each) is approximated as the optical axis of the eye. For example, see the method in Non-patent Literature 3 which will be described later. Since the pupil centerline is also used as the optical axis of the eye (optic axis of Gullstrand), the κ angle can be easily estimated based on at least the amount of displacement of the two A-scans (details will be described later in the example). Since the α angle is an angle between an optical axis and a visual axis of the eye, it is possible that the κ angle obtained as described above can be regarded as substantially the same as the α angle.
Non-patent Literature 3: Hyung-Jin Kim, et al. “Full ocular biometry through dual-depth whole-eye optical coherence tomography” Biomedical Optics Express Vol. 9, Issue 2, pp. 360-372 (2018)

Here, in a case where the cornea and the crystalline lens in the wide-area OCT data include at least a corneal luminous point and a crystalline lens luminous point, a straight line passing through the corneal luminous point and the crystalline lens luminous point can be obtained as the optical axis of the eye (and pupil centerline). Thus, in the wide-area OCT data, it is possible to specify the optical axis of the eye (and pupil centerline) even in a case where it is difficult to detect curves of the cornea and the crystalline lens. Therefore, the optical axis of the eye (and the pupil centerline) itself and the tilt information based on the optical axis of the eye can be easily acquired from one-shot wide-area OCT data captured with the focus on the fundus side.

In a case where the curves of the cornea and the lens are detectable in the wide-area OCT data, each curvature center may be obtained based on curve shapes of the cornea and the crystalline lens, and the optical axis of the eye may be obtained based on each curvature center.

Further, in a case where wide-range anterior segment OCT is included in the wide-area OCT data, by using information on the iris (more preferably corner angle) and the corneal, the pupil centerline can be obtained more strictly anatomically as a straight line which passes through a pupil center and is perpendicular to the cornea. The pupil centerline obtained in this manner may be used in calculating the κ angle.

Further, by analyzing the wide-area OCT data, various reference axes of the eye or information based on the reference axes may be acquired. The reference axis may be, for example, any one of the visual axis, a gaze line, a line of sight, the pupil centerline, the axis of the eye, or the like. Further, it is believed that this information can be used to obtain physiological squint angles other than the α and κ angles.

### Example

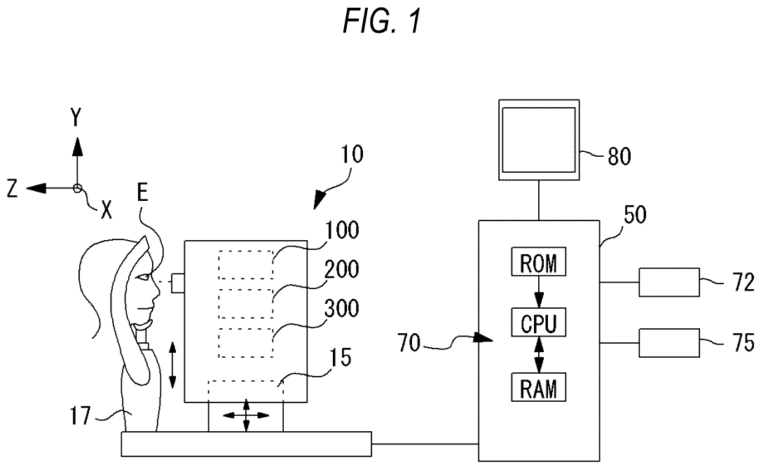

Hereinafter, an OCT system (optical coherence tomography system) illustrated in FIGS. 1 and 2 will be described as an example.

The OCT system of the present example switches measurement ranges of a subject eye E. Imaging modes are switched to image a fundus, an anterior segment, and a whole-eye of the subject eye in each imaging mode.

As illustrated in FIG. 1, the OCT system according to the example includes at least an optical unit 10 and a control unit 50 corresponding to a computer of the present example. In the present example, the optical unit 10 and the control unit 50 are integrated as an OCT device. The OCT system (OCT device) according to the present example has a basic configuration of wavelength-swept OCT (SS-OCT).

The optical unit 10 has a light guide optical system 150. Further, the optical unit 10 in the present example includes a fundus observation optical system 200 and an anterior segment observation optical system 300.

The optical unit 10 is three-dimensionally movable by an XYZ movement unit 15. In the present example, the XYZ movement unit 15 is driven and controlled by an arithmetic controller 70. In the present example, a three-dimensional position of the optical unit 10 with respect to the subject eye E is adjusted by three-dimensionally moving the optical unit 10 by the XYZ movement unit 15. Thus, the three-dimensional position of the optical unit 10 is aligned with the subject eye E. Further, a face of a subject person is supported by a face support unit 17. A support position of the face by the face support unit 17 is movable in an upward-downward direction.

The control unit 50 is a computer in the present example, and includes at least the arithmetic controller (processor) 70 that controls the entire OCT system. The arithmetic controller 70 is configured with, for example, a CPU, a memory, and the like. As an example, in the present example, the arithmetic controller 70 also serves as an image processor in the OCT system.

In addition, the OCT system may be provided with a storage unit (memory) 72, an input interface (operation unit) 75, a monitor 80, and the like. Each unit is connected to the arithmetic controller 70.

Various programs for controlling an operation of the OCT device, initial values, and the like may be stored in the memory 72. For example, a hard disk drive, a flash ROM, a USB memory detachably mounted on the OCT device, and the like can be used as the memory 72. Further, in the memory 72, various types of information related to imaging may be stored in addition to an OCT image generated from OCT data. The monitor 80 may display the OCT data (OCT image).

<OCT Optical System>

Next, an OCT optical system 100 in the present example will be described with reference to FIG. 2. The OCT optical system 100 causes the light guide optical system 150 to guide measurement light to the subject eye E. The OCT optical system 100 guides reference light to a reference optical system 110. The OCT optical system 100 causes a detector (light reception element) 120 to receive spectrum interference signal light acquired by interference between the measurement light reflected by the subject eye E and the reference light.

In the present example, the OCT optical system 100 uses an SS-OCT method. In this case, the OCT optical system 100 has a wavelength-swept light source as an OCT light source 102. Further, the OCT optical system 100 has a point detector as the detector 120.

In the wavelength-swept light source, an emission wavelength is swept in time. The OCT light source 102 may be a VCSEL-type wavelength-swept light source. The VCSEL-type wavelength-swept light source includes a VCSEL responsible for laser oscillation and an MEMS that realizes high-speed scanning. A device capable of changing a sweep frequency (scan rate) is used as the VCSEL-type wavelength-swept light source in the present example. For example, the OCT light source 102 in the present example can be varied to a plurality of sweep frequencies in a range from at least 20 kHz (second frequency in the present example) to 400 kHz (first frequency in the present example).

In the present example, the detector 120 is a balanced detector that performs balanced detection using a plurality of (for example, two) detectors. The arithmetic controller samples an interference signal of return light of the reference light and the measurement light according to a change in emission wavelength of the wavelength-swept light source, and obtains OCT data of the subject eye based on the interference signal at each wavelength obtained by the sampling. In the present example, a sampling period is appropriately adjusted such that a measurement range in the depth direction is changed according to the sweep frequency of the OCT light source 102.

A coupler (splitter) 104 is used as a first light splitter, and splits light emitted from the OCT light source 102 into a measurement optical path and a reference optical path. For example, the coupler 104 guides the light from the OCT light source 102 to an optical fiber 152 on a measurement optical path side, and also guides the light to the reference optical system 110 on a reference optical path side.

<Light Guide Optical System>

The light guide optical system 150 is provided to guide measurement light to the subject eye E. The light guide optical system 150 may be provided sequentially with, for example, an optical fiber 152, a collimator lens 153, a focusing lens 155, an optical scanner 156, and an objective lens system 158 (objective optical system in the present example). In this case, the measurement light is emitted from an emission end of the optical fiber 152, and converted into a parallel beam by the collimator lens 153. After that, the parallel beam goes to the optical scanner 156 via the focusing lens 155. The focusing lens 155 can be displaced along an optical axis by a drive unit (not illustrated), and is used to adjust a condensing state. The subject eye E is irradiated with the light passing through the optical scanner 156, via the objective lens system 158. In the present example, a pivot point P is formed at a conjugate position with the optical scanner 156 with respect to the objective lens system 158 (provided in the device main body). In the present example, as will be described later, a position of the pivot point P with respect to at least any one of the subject eye E or the optical system of the device is changed, according to a measurement range (in other words, imaging portion) of the subject eye E in the depth direction.

The optical scanner 156 may scan the measurement light in an XY direction (transverse direction) on a tissue of the subject eye E. In the present example, the optical scanner 156 is, for example, two galvano mirrors, and a reflection angle thereof is freely adjusted by a drive mechanism. A luminous flux emitted from the OCT light source 102 changes its reflection (traveling) direction, and is scanned in any direction on the tissue of the subject eye E. As the optical scanner 156, for example, a reflection mirror (galvano mirror, polygon mirror, and resonant scanner), an acoustic optical element (AOM) that changes the traveling (deflection) direction of light, or the like may be used.

Scattered light (reflected light) from the subject eye E by the measurement light travels back along a path of light projection, is incident on the optical fiber 152, and reaches the coupler 104. The coupler 104 guides the light from the optical fiber 152 into an optical path toward the detector 120.

<Reference Optical System>

The reference optical system 110 generates reference light. The reference light is synthesized with the reflected light from the subject eye E of the measurement light. The reference light passing through the reference optical system 110 is synthesized with light from a measurement optical path by the coupler 148, and interferes with the light. The reference optical system 110 may have a Michaelson type or a Mach Zenda type.

The reference optical system 110 illustrated in FIG. 2 is formed with a transmissive optical system as an example. In this case, the reference optical system 110 guides light from the coupler 104 to the detector 120 by transmitting the light rather than returning the light. The reference optical system 110 is not limited to this, for example, may be formed by a reflective optical system, and the light from the coupler 104 may be guided to the detector 120 by being reflected by the reflective optical system. In the present example, an optical path length difference adjuster 145 and a polarization adjuster 147 are disposed on an optical path from the coupler 104 to the detector 120.

The optical path length difference adjuster 145 is used to adjust an optical path length difference between the measurement light and the reference light. When acquiring OCT data, it is necessary to previously adjust the optical path length difference between the measurement light and the reference light according to at least a depth position of an imaging target (portion of the subject eye E). In the present example, a mirror 145a having two orthogonal surfaces is provided on a reference optical path. An optical path length of the reference optical path can be increased or decreased by moving the mirror 145a in an arrow direction by an actuator 145b. Of course, a configuration for adjusting the optical path length difference between the measurement light and the reference light is not limited to this. For example, in the light guide optical system 150, by integrally moving the collimator lens 153 and the coupler, an optical path length of the measurement light may be adjusted, and as a result, the optical path length difference between the measurement light and the reference light may be adjusted.

In the present example, the polarization adjuster 147 adjusts polarization of the reference light. The polarization adjuster 147 may be disposed on the measurement optical path.

<Acquisition of Depth Information>

The arithmetic controller 70 performs processing (Fourier analysis) on a spectral signal detected by the detector 120 to obtain OCT data of the subject eye.

The spectral signal (spectral data) may be rewritten as a function of wavelength k, and converted into a function I (k) at equal intervals with respect to wavenumber k (=27c/k). Alternatively, it may be acquired as the function I (k) at equal intervals with respect to wavenumber k from a beginning (K-CLOCK technique). The arithmetic controller 70 may obtain OCT data in a depth (Z) domain by Fourier transforming the spectral signal in a wavenumber k space.

Further, the information after the Fourier transform may be represented as a signal including a real number component and an imaginary number component in a Z space. The arithmetic controller 70 may obtain the OCT data by obtaining absolute values of the real number component and the imaginary number component in the signal in the Z space.

<Description of Operation>

Next, an operation of the OCT device according to the example will be described based on flowcharts.

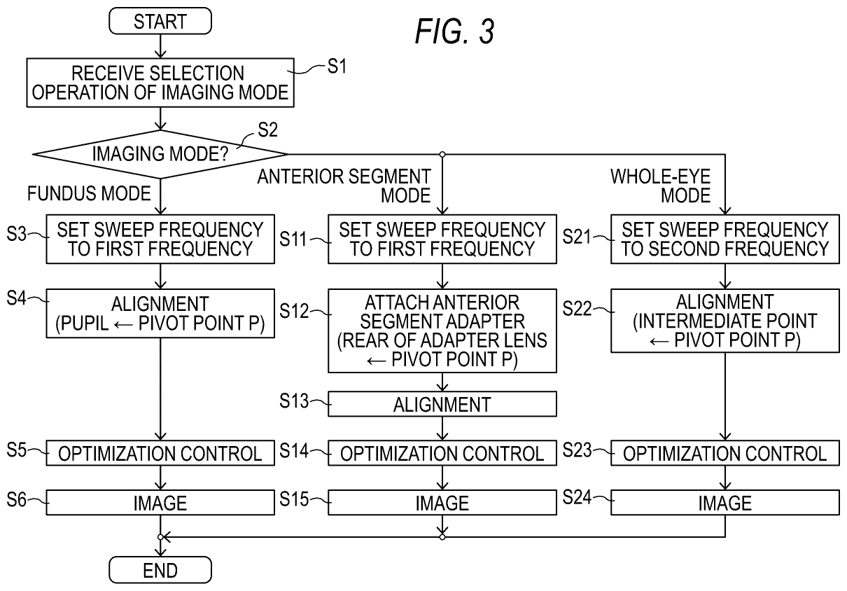

First, a flow up to imaging will be described with reference to the flowchart in FIG. 3.

<S1 and S2: Setting of Imaging Mode>

In the present example, an imaging mode is set in advance based on a selection operation (S1 and S2). Here, any one of three types of imaging modes corresponding to a measurement range of the subject eye E is set based on the selection operation. A fundus mode, an anterior segment mode, and a whole-eye mode can be set as the imaging mode. The selection operation of the imaging mode may be input via a setting screen. Further, a scan pattern, an imaging type, and the like may be set at this time.

<Fundus Mode>

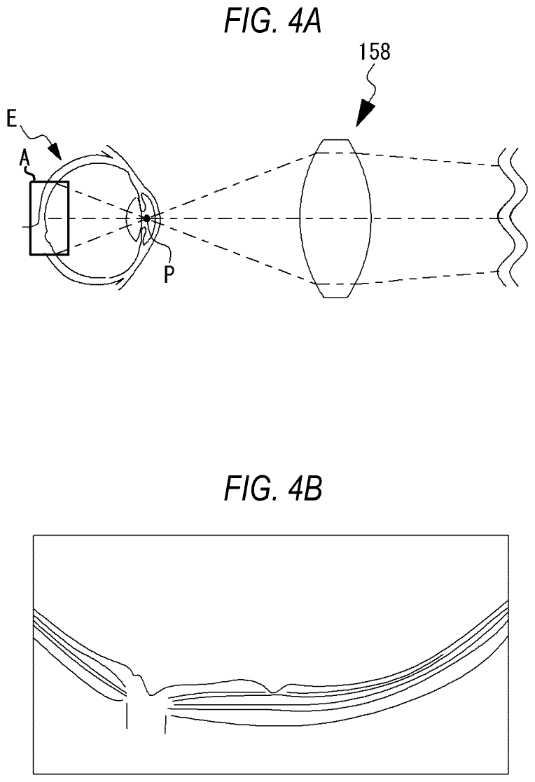

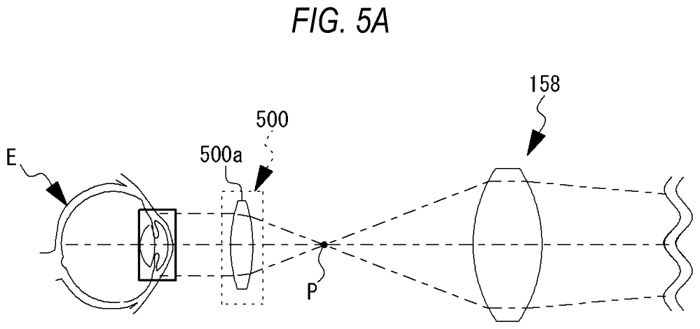

In a case where a fundus of the subject eye is in the measurement range, the fundus mode is selected (fundus mode in S2). As illustrated in FIG. 4A, in the fundus mode, the measurement range is approximately several millimeters around the fundus. In this case, the arithmetic controller 70 sets a sweep frequency of the OCT light source 102 to a first frequency (400 kHz in the present example) (S3). Thus, the measurement range in the depth direction is adjusted to approximately several millimeters.

Further, in the present example, an alignment state and a state of each unit of the OCT optical system 100 are adjusted according to the measurement range (S4 and S5).

First, a three-dimensional position of the optical unit 10 with respect to the subject eye E is guided to a position appropriate for imaging the fundus (S4). That is, as illustrated in FIG. 4A, the three-dimensional position is guided such that the pivot point P is arranged in an anterior segment of the subject eye (more specifically, a pupil center). By arranging the pivot point P in the anterior segment of the subject eye, measurement light reaches the fundus without being eclipsed by an iris. The measurement light is scanned around the pivot point P according to the operation of the optical scanner 156. At this time, since a position of the pivot point P substantially coincides with a position of a principal point in the ocular optical system, it is less likely to be affected by the refraction of a translucent body of the subject eye E. Therefore, in the fundus mode, a wide range of the fundus can be imaged.

At a time of alignment adjustment, for example, after having a subject person gaze at a fixation target in advance, based on an anterior segment observation image acquired via the anterior segment observation optical system 300, the arithmetic controller 70 drives and controls the XYZ movement unit 15 to adjust a positional relationship between the subject eye and the optical unit 10. At a position at which the alignment adjustment is completed, the fundus observation optical system 200 acquires a front image of the fundus as an observation image.

After the alignment is completed, acquisition of the observation image via the fundus observation optical system 200 and a display of the observation image on the monitor are started. At the same time, the arithmetic controller 70 acquires OCT image of the fundus at any time via the OCT optical system 100.

Next, optimization control of an imaging condition is performed (S5). The state of each portion of the OCT optical system 100 (that is, the imaging conditions) is adjusted according to the fundus portion that is the measurement range. As a result, the fundus portion can be observed with high sensitivity and high resolution by the OCT optical system 100. In the present example, as an example of optimization control in the OCT optical system 100, optical path length adjustment, focus adjustment, and polarization state adjustment (polarizer adjustment) are executed. In the present example, in the polarizer adjustment, the polarization adjuster 147 is driven and controlled (the same applies to the anterior segment mode and the whole-eye mode) based on an output signal output from the light reception element 120 such that a polarization state of the measurement light and the reference light coincide with each other (here, a stronger interference signal is obtained).

For example, the optimization control is started by operating an optimization start button (Optimize button) (not illustrated) as a trigger. Thus, the optical path length difference is adjusted such that the fundus image is detected within a predetermined section from a zero delay position in the OCT data. After adjusting the optical path length difference, the focusing lens is driven according to a position at which the fundus image is detected on the OCT data, and a focus position is adjusted. Meanwhile, in detecting an optimum focus position, instead of using the OCT data, or additionally, in conjunction with focus adjustment in the fundus observation optical system 200 using the observation image, focus adjustment in the OCT optical system 100 may be performed.

In the present example, when an examiner presses an imaging switch (not illustrated) after the optimization is completed, OCT data of the fundus is imaged (captured) via the OCT optical system 100. At this time, the OCT data may be imaged by using any one of a plurality of predetermined scan patterns. FIG. 4B illustrates a B-scan image of the fundus as an example of the OCT data of the fundus obtained by the imaging. Meanwhile, the present example is not limited to this, and volume data may be captured. The imaged OCT data may be stored (saved) in a memory of the device in association with a scanning position and identification information indicating a date and time of the imaging. Thus, the imaged OCT data is acquired by the arithmetic controller 70 as a captured image.

In addition, in the fundus mode, OCT data of a peripheral portion of the fundus may be captured. In this case, a fixation position is changed with respect to the case of imaging the center portion of the fundus, and the OCT data is captured after optimization control is executed on the peripheral portion of the fundus.

<Anterior Segment Mode>

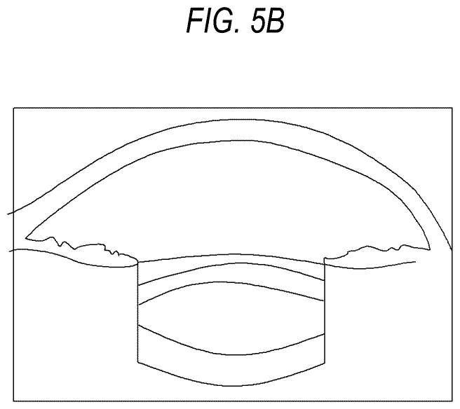

In a case where the anterior segment of the subject eye is in the measurement range, the anterior segment mode is selected (anterior segment mode in S2). As illustrated in FIG. 5A, in the anterior segment mode, the measurement range is approximately several millimeters of the anterior segment. In this case, the arithmetic controller 70 sets the sweep frequency of the OCT light source 102 to the first frequency (400 kHz in the present example) (S11). Thus, the measurement range in the depth direction is adjusted to approximately several millimeters. Thus, in the present example, the sweep frequency is the same between the fundus mode and the anterior segment mode, and the sweep frequencies in each mode may be different from each other.

Further, in the present example, in a case of the anterior segment mode, an anterior segment adapter 500 is attached to the device (S12). By attaching the anterior segment adapter 500, an adapter lens 500a is inserted between the objective lens system 158 of the device main body and the subject eye E (in the present example, between the pivot point P and the subject eye E). Thus, the pivot point P substantially coincides with a focal position of the adapter lens 500a. As a result, measurement light is emitted substantially parallel to an optical axis via the adapter lens 500a. That is, an optical system telecentric to the object side is formed by the objective lens system 158 of the device main body and the adapter lens 500a as an objective optical system in the anterior segment mode.

Thus, since a magnification change of the captured image due to a position change of the subject eye E is reduced, it is easy to accurately measure an intraocular distance based on the captured anterior segment tomographic image. In addition, since the object side is telecentrically irradiated with the measurement light, a distortion of the tomographic image due to the deviation of the subject eye E in an operation distance direction is less likely to occur. Further, by being telecentric on the object side, the measurement light can be easily emitted on a portion distant from a visual axis of the subject eye E, and return light (reflected light or backscattered light) from the anterior segment can be collected more efficiently. Therefore, it is possible to suppress a decrease in luminance in the peripheral portion of the image.

Next, alignment adjustment is performed (S13). At this time, for example, alignment adjustment between the subject eye E and the optical unit 10 may be performed based on an observation image acquired by the fundus observation optical system 200.

Next, optimization control of an imaging condition is performed (S14). The state of each portion of the OCT optical system 100 is adjusted according to the anterior segment, which is the measurement range. As a result, the anterior segment can be observed with high sensitivity and high resolution by the OCT optical system 100. In a case where the anterior segment is imaged, an individual difference of the subject eye E is less problematic than a case where the fundus is imaged, so that the optical path length, the focus position, and the like may be adjusted to substantially constant values regardless of the subject eye E, for example.

Next, when the examiner presses an imaging switch (not illustrated), OCT data of the anterior segment (see FIG. 5B) is imaged (captured) via the OCT optical system 100, and stored (saved) in the memory of the device.

In the anterior segment mode, the OCT data of the anterior segment including a corner angle to a sclera may be captured. In this case, the imaging may be executed after shifting the alignment position in the XY direction with respect to the visual axis. Further, the OCT data of the anterior segment including front and back surfaces of a crystalline lens may be captured.

<Whole-Eye Mode>

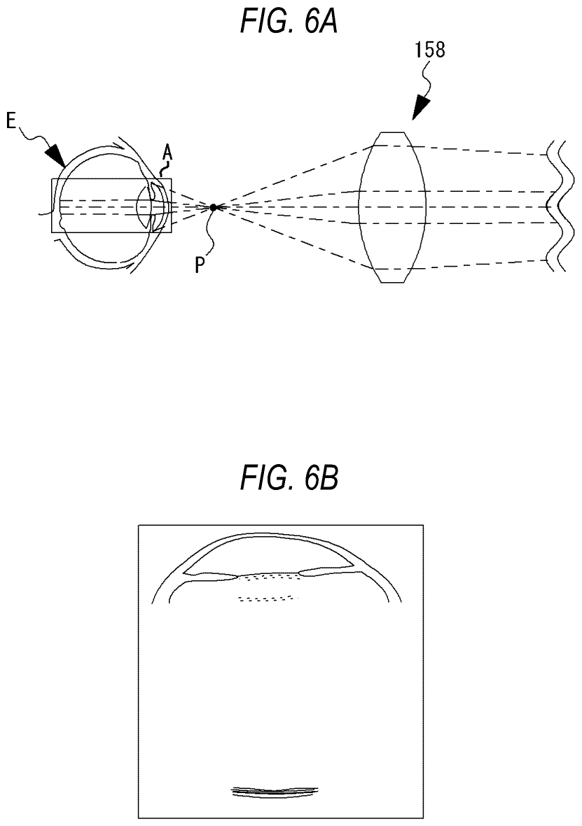

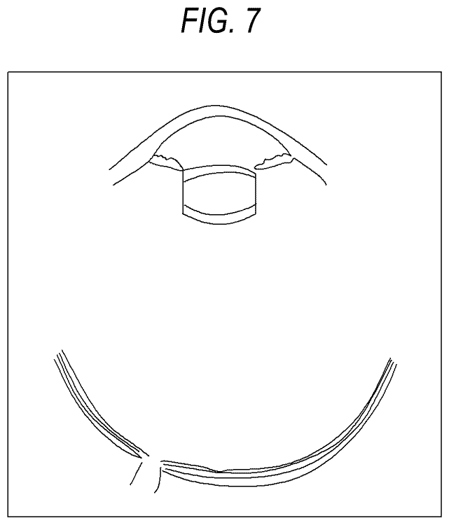

In a case where the whole-eye (here, the anterior segment and the fundus) of the subject eye is set as the measurement range, the whole-eye mode is selected (whole-eye mode in S2). In the present example, as illustrated in FIG. 6A, in the whole-eye mode, the whole-eye including the anterior segment and the fundus is in the measurement range. In this case, the arithmetic controller 70 sets the sweep frequency of the OCT light source 102 to a second frequency (20 kHz in the present example) (S21). Thus, the measurement range in the depth direction is adjusted to approximately 30 mm.

Further, in the present example, the alignment state and the state of each portion of the OCT optical system 100 are adjusted according to the measurement range (S22 and S23).

First, the three-dimensional position of the optical unit 10 with respect to the subject eye E is guided to a position appropriate for imaging the whole-eye (S22). That is, as illustrated in FIG. 6A, the three-dimensional position is guided such that the pivot point P is arranged between the subject eye and the objective lens system 158. By arranging the pivot point P between the subject eye and the objective lens system 158, it is possible to emit the measurement light to the cornea, the fundus, and a part of the iris. Therefore, in the whole-eye mode of the present example, OCT data including at least the cornea, the fundus, and the part of the iris can be acquired in one shot. From such whole-eye OCT data, the positional relationship between the fundus and the anterior segment can be appropriately specified.

In the whole-eyeball mode of the present example, alignment adjustment is performed in at least the XY direction based on an anterior segment observation image acquired via the anterior segment observation optical system 300. The alignment adjustment in the Z direction may be performed based on an alignment index image projected onto the cornea of the subject eye from an alignment projection optical system (not illustrated). Alternatively, it may be adjusted based on OCT data acquired by the OCT optical system 100. The alignment may be automatically adjusted by the arithmetic controller 70.

After the alignment is completed, acquisition of the observation image via the fundus observation optical system 200 and a display of the observation image on the monitor 80 are started. At the same time, the arithmetic controller 70 acquires whole-eye OCT image at any time via the OCT optical system 100.

In optimization control of the imaging condition in the whole-eyeball mode (S23), the state of each portion of the OCT optical system 100 (that is, the imaging condition) is adjusted according to the measurement range. The optical path length difference is adjusted such that the images of the anterior segment and the fundus are detected within a predetermined section from the zero delay position in the OCT data. Further, the focus position may be adjusted by driving the focusing lens according to a position at which the images of the anterior segment and the fundus are detected on the OCT data. The focus position may be adjusted near the image position of the anterior segment or the fundus, or may be adjusted midway between the anterior segment and the fundus.

In the present example, after the optimization is completed, when the examiner presses an imaging switch (not illustrated), the OCT data of the whole-eye (see FIG. 6B) is imaged (captured) via the OCT optical system 100, and saved.

<Curvature Correction>

In each OCT data illustrated in FIGS. 4B, 5B, and 6B, an image is formed by arranging A-scan data parallel to the scanning direction (linear direction) of the measurement light. The fundus OCT data and the whole-eye OCT data are expressed on polar coordinates with the pivot point P as a center, so that a curvature of the image resulting from scanning with the pivot point P as the center is corrected. The corrected OCT data is expressed as a more accurate image with respect to a shape of the actual subject eye. When converting the fundus OCT data and the whole-eye OCT data into the polar coordinates, the curvature of the image of the tissue in the eyeball may be corrected in consideration of refraction of the measurement light by the translucent body of the subject eye. At this time, for example, the curvature may be corrected in a ray-tracking manner.

Further, for the anterior segment OCT data, when the emission of the measurement light is not completely parallel to the optical axis, the curvature of the image may be corrected according to a tilt of the light beam with respect to the optical axis of the OCT optical system 100 during each A-scan.

<Collage>

In the whole-eye OCT data, the subject eye is captured in a wide range in the depth direction. Meanwhile, in the transverse direction, each portion of the anterior segment OCT data and the fundus OCT data can be captured in a wider range. Therefore, by synthesizing the anterior segment OCT data and the fundus OCT data with the whole-eye OCT data, a wide-area OCT image is generated. The curvature correction may be performed for each OCT data to be synthesized. The wide-area OCT image may be generated by performing position alignment and synthesizing the images related to a feature portion included in each image. The position alignment between the images may be rigid registration or non-rigid registration.

In the whole-eye OCT data, the positional relationship between the fundus and the anterior segment can be appropriately specified, so that the local OCT data of the anterior segment or the fundus can be appropriately synthesized (collaged) with the whole-eye OCT data. That is, it is possible to generate a wide-area OCT image in which the actual positional relationship between the fundus and the anterior segment is reflected.

For example, in order to obtain the OCT image illustrated in FIG. 7, a plurality of pieces of OCT data for each of the anterior segment and the fundus may be synthesized with whole-eye OCT data. More specifically, regarding the anterior segment, each of OCT data of the anterior segment including the corner angle to the sclera and OCT data of the anterior segment including the front and back surfaces of the crystalline lens may be synthesized with the whole-eye OCT data. Further, for example, regarding the fundus, each of OCT data of the fundus imaged with the optical axis of the OCT optical system 100 and the fixation optical axis being aligned and OCT data of the fundus with the optical axis of the OCT optical system 100 and the fixation optical axis being tilted may be synthesized with the whole-eye OCT data.

<Analysis Processing>

Various types of analysis processing may be performed on whole-eye OCT data (or the above-described synthesized image based on the whole-eye OCT data). For example, analysis processing related to eye dimension information may be performed. Any one of various types of eye dimension information such as a corneal thickness, an anterior chamber depth, an axial length of the eye, an angle of an anterior chamber angle, or the like may be obtained by the analysis processing.

Further, based on the whole-eye OCT data (or the above-described synthesized image based on the whole-eye OCT data), information on the positional relationship of each portion may be acquired. In particular, information representing the tilt of the anterior segment relative to the fundus may be acquired.

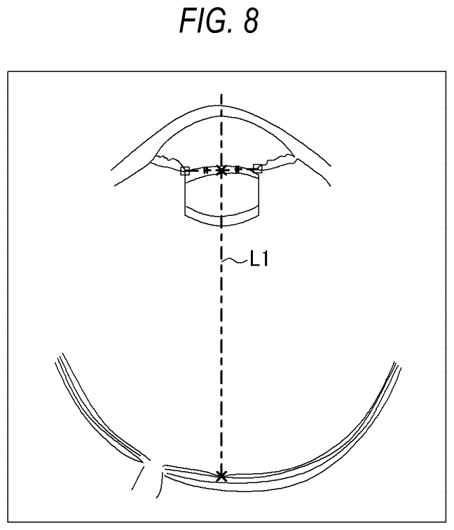

As illustrated in FIG. 8, as a specific example, in the present example, a straight line connecting the fovea centralis on the fundus and the pupil center is detected as an axis L1 based on the whole-eye OCT image (or the above-described synthesized image based on the whole-eye OCT data). In addition, a tilt angle of the axis L1 with respect to the optical axis of the OCT optical system 100 is further derived. Positional information itself of the axis L1 and the tilt angle of the axis L1 with respect to the optical axis of the OCT optical system 100 are obtained as information representing the tilt of the anterior segment with respect to the fundus. In this manner, since the whole-eye OCT data includes the positional information on the cornea, the iris, and the fundus, it is possible to appropriately specify the positional relationship between the fundus and the anterior segment. As a result, for example, it is possible that IOL is appropriately positioned during IOL prescriptions.

### Modification Example

Although the present disclosure is described above based on the embodiments and examples, the present disclosure is not necessarily limited to this, and various modification examples can be made.

For example, in the OCT device of the above-described example, by changing the sweep frequency, the fundus mode, the anterior segment mode, and the whole-eye mode can be switched. Meanwhile, the OCT device is not necessarily limited to this, and may be capable of performing imaging in at least the whole-eye mode.

For example, the OCT device does not need to have two or more optical scanners. The OCT device may be capable of scanning measurement light in only one direction with a single optical scanner.

<Measurement Method Advantageous for Acquisition of Physiological Squint Angle>

In the above-described example, in the whole-eyeball mode, as illustrated in FIG. 6A, the three-dimensional position is guided such that the pivot point P is arranged between the subject eye and the objective lens system 158. In this state, further, wide-area OCT data acquired after the three-dimensional position is guided such that the corneal luminous point, the crystalline lens luminous point, and the fovea centralis on the fundus are included in a tomographic image may be acquired. For example, while the three-dimensional position is being guided, in the tomographic image that is acquired at any time, the presence or absence of the corneal luminous point and the crystalline lens luminous point may be detected, and the three-dimensional position may be automatically guided to a position at which the corneal luminous point and the crystalline lens luminous point are detected. In addition, while the three-dimensional position is being guided, the tomographic image acquired at any time may be displayed such that the examiner can manually adjust the three-dimensional position.

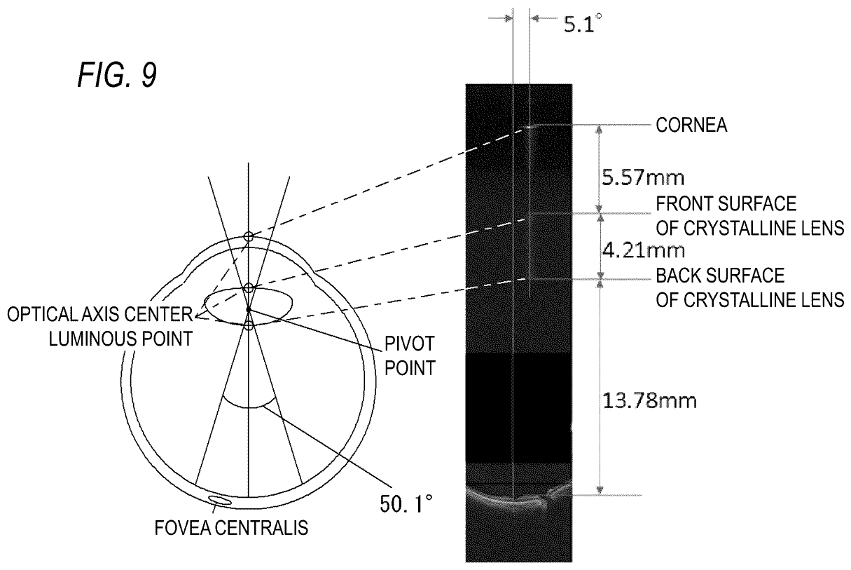

An example of OCT data acquired at this time is illustrated as a tomographic image in FIG. 9. The tomographic image illustrated in FIG. 9 may be one after full-range processing.

As a result of arranging the pivot point of the measurement light on a side of the subject eye (preferably, the anterior segment) of the objective lens, the fundus is scanned over a wide range. Therefore, as illustrated in the tomographic image in FIG. 9, it can be seen that there is a sufficient amount of information in the transverse direction of the fundus to specify the optical features of the subject eye.

Further, in the tomographic image illustrated in FIG. 9, a luminous spot occurs in the cornea and the crystalline lens. The luminous spot is considered to occur at a center of the optical axis of the subject eye as illustrated in the schematic diagram.

The κ angle, which is an example of a physiological squint angle, is defined as an angle between the visual axis of the subject eye and the pupil centerline, and the visual axis is an A-scan passing through the fovea centralis on the retina. Further, the A-scan corresponding to the optical axis of the eye is specified from information on the cornea and the crystalline lens in the OCT data. By considering the optical axis of the eye and the pupil centerline, the κ angle can be estimated based at least on a displacement amount of the two A-scans.

That is, since the number of A-scans occupying an angle of view is generally known, an estimated value of the κ angle can be obtained by converting the displacement amount of two A-scans into the angle of view.

In this case, the scanning direction of the wide-area OCT data is reversed between the front side and the back side of the pivot point of the measurement light. Therefore, the κ angle may be obtained as described above after performing processing for correcting the scanning direction on the image or the calculation.

Further, as illustrated in FIG. 9, eye dimension information such as a corneal thickness, an anterior chamber depth, and an axial length of the eye can be obtained from OCT data including the cornea to the fundus. The κ angle is used as an index of whether toric IOL prescriptions are appropriate for the subject eye, and the eye dimension information is used for IOL calculation. Therefore, information used in IOL prescriptions can be appropriately acquired, so that it is possible that more appropriate IOL can be prescribed.

The OCT device according to the present disclosure can also be expressed as follows.

For example, there is provided a first ophthalmic image processing program executed by a processor of an ophthalmic computer, causing the ophthalmic computer to execute: an acquisition step of acquiring wide-area OCT data of a subject eye including at least OCT data of an anterior segment and OCT data of a fundus acquired in a state where a pivot point of measurement light is arranged closer to a side of the subject eye than an objective optical system; and an analysis processing step of acquiring a physiological squint angle of the subject eye by analyzing the wide-area OCT data are executed by the ophthalmic computer.

In a second ophthalmic image processing program according to the first ophthalmic image processing program, the wide-area OCT data is obtained by capturing the OCT data of the anterior segment and the OCT data of the fundus in one shot.

In a third ophthalmic image processing program according to the second ophthalmic image processing program, the wide-area OCT data is captured in a state in which the pivot point of the measurement light is adjusted to the anterior segment of the subject eye.

In a fourth ophthalmic image processing program according to the third ophthalmic image processing program, the wide-area OCT data includes a cornea, a crystalline lens, and a fovea centralis on the fundus, and in the analysis processing step, the physiological squint angle is determined based on at least a positional relationship between the cornea, the crystalline lens, and the fovea centralis on the fundus in the OCT data.

In a fifth ophthalmic image processing program according to the fourth ophthalmic image processing program, the cornea and the crystalline lens in the wide-area OCT data include at least a corneal luminous point and a lens luminous point, and in the analysis processing step, the physiological squint angle is obtained based on at least a positional relationship between the corneal luminous point, the crystalline lens luminous point, and the fovea centralis on the fundus in the OCT data.

A first OCT device executes any one of the first to fifth ophthalmic image processing programs.

A second OCT device aligns an OCT optical system for acquiring the wide-area OCT with respect to the subject eye such that the wide-area OCT data includes the corneal luminous point and the crystalline lens luminous point in the first OCT device.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An OCT device comprising:
an OCT optical system including:
a light splitter that splits light from an OCT light source into measurement light and reference light; and
a detector that detects a spectral interference signal between the measurement light guided to a subject eye and the reference light;
an arithmetic controller that acquires OCT data based on a signal from the detector, and controls at least the OCT optical system to execute an OCT data acquisition operation;
a light guide optical system including at least:
an optical scanner that scans a tissue of the subject eye with the measurement light; and
an objective optical system that is disposed between the optical scanner and the subject eye, and forms a pivot point around which the measurement light passing through the optical scanner is pivoted; and
an alignment adjuster that adjusts a three-dimensional position of the light guide optical system with respect to the subject eye,
wherein the arithmetic controller guides the three-dimensional position such that the pivot point is arranged at a target position set between the subject eye and the objective optical system, and further executes the OCT data acquisition operation at the target position.

2. The OCT device according to claim 1, further comprising:
a second adjuster that adjusts a measurement range in a depth direction in the OCT data,
wherein in a case where the OCT data acquisition operation is executed at the target position, the arithmetic controller controls the second adjuster such that the measurement range includes a cornea to a fundus of the subject eye.

3. The OCT device according to claim 2, further comprising:
a wavelength-swept light source that is the OCT light source; and
a converter that samples the spectral interference signal output from the detector, and converts the spectral interference signal from an analog signal to a digital signal,
wherein the second adjuster is at least any one of the wavelength-swept light source or the converter, and
the arithmetic controller controls a sweep frequency of the wavelength-swept light source or a sampling rate of the spectral interference signal in the converter such that the measurement range is longer than an axial length of the subject eye.

4. The OCT device according to claim 2,
wherein the second adjuster includes at least an optical path length difference adjuster that adjusts an optical path length difference between the measurement light and the reference light.

5. The OCT device according to claim 1, further comprising:
a focus adjuster that adjusts a focus position of the measurement light,
wherein at the target position, the arithmetic controller adjusts the focus position of the measurement light to a first focus position to acquire first OCT data, further adjusts the focus position to a second focus position different from the first focus position acquires second OCT data, and acquires synthetic OCT data by synthesizing the first OCT data and the second OCT data.

6. The OCT device according to claim 1,
wherein the arithmetic controller executes the OCT data acquisition operation at the target position such that B-scan data of the OCT data includes a cornea, an iris, and a fundus of the subject eye.

7. The OCT device according to claim 6,
wherein the arithmetic controller analyzes the B-scan data to acquire information representing a tilt of an anterior segment with respect to the fundus.

8. The OCT device according to claim 7,
wherein the arithmetic controller acquires a physiological squint angle of the subject eye as the information representing the tilt of the anterior segment with respect to the fundus.

9. The OCT device according to claim 8,
wherein the cornea and a crystalline lens in the OCT data include at least a corneal luminous point, and a crystalline lens luminous point, and
the arithmetic controller obtains the physiological squint angle based at least on a positional relationship between the corneal luminous point, the crystalline lens luminous point, and a fovea centralis on the fundus in the B-scan data.

10. The OCT device according to claim 7,
wherein the arithmetic controller analyzes the B-scan data to acquire the information representing the tilt of the anterior segment with respect to the fundus, based on a positional relationship between a pupil center based on an end portion of the iris and a fovea centralis on the fundus.
